(19) United States
(12) Patent Application Publication
(10) Pub. No.: US 2004/0152968 A1
(43) Pub. Date: Aug. 5, 2004

(54) MRI-compatible surgical instruments

(76) Inventors: Alfred A. Iversen, Wayzata, MN (US); Eric F. Caille, Minnetonka, MN (US); Deron J. Singer, Shakopee, MN (US)

Correspondence Address: Anthony G. Eggink, Anthony G. Eggink & Associates, 3100 First National Bank Building, 332 Minnesota Street, Saint Paul, MN, 55101, US

(21) Appl. No.: 10/761,450

(22) Filed: Jan. 20, 2004

Related U.S. Application Data
(60) Provisional application No. 60/440,748, filed on Jan. 17, 2003.

(51) Int. Cl. A61B 5/05

(52) U.S. Cl. 600/411

(57) ABSTRACT

Single-use, MRI compatible surgical and microsurgical instruments for neurosurgery, orthopedic surgery and other surgical uses. The surgical instruments are constructed of polymeric materials, graphite, titanium, nitonol compositions and combinations thereof. The instruments may have transparent polymeric portions, reference points on the handle and may have non-reflective surfaces. The instruments are constructed and arranged for use in interventional MRI suites.

This application claims the benefit of U.S. Provisional Application No. 60/440,748, filed on Jan. 17, 2003.

### BACKGROUND OF THE INVENTION

The present invention relates generally to surgical and microsurgical instruments. Particularly, the invention relates to single use, disposable surgical instruments which are MRI (magnetic resonance imaging) compatible, easy to use and economical.

Presently surgical and microsurgical instruments are typically constructed of stainless steel and are provided to be reusable. These prior art instruments are not MRI compatible and cannot be used in interventional MRI suites due to their ferrous compositions. Additionally, contact with non-MRI compatible instruments can potentially contaminate the MRI compatible instruments with ferrous substances.

Prior art surgical instruments are also designed and constructed to be reused after sterilization. However, recent events suggest that sterilization may not destroy all harmful contaminants from the instruments. For example, it has been found that varient Jakob-Creutzfeld disease may be spread via the reuse of surgical instruments previously used on a patient having the disease even though the instruments have been properly sterilized via radiation, autoclaving or like sterilization procedures. A need therefore exists for surgical instruments that are specifically constructed and designed for one time use.

Most reusable surgical instruments are expensive and may be damaged in surgery by power tools or simply by handing during the process. Further, prior art surgical instruments may block the view of the surgical site and may yield a glare due to reflecting light. As far as is known, no surgical instruments have been proposed or sold in the marketplace having a transparent view of the surgical site beneath the instrument location.

A need exists for surgical and microsurgical instruments that are MRI-compatible, lightweight, cost efficient and designed for single use. The present invention provides surgical instruments for neurosurgery, orthopedic surgery and other surgical uses and which include micro-probes, dissectors, suction instruments, micro-ring curettes, probes and other surgical devices and apparatus, for example, which are used in dissection, probing, cutting, curing, the removal of fluid and matter from a surgical site and other surgical applications. As far as is known, no such MRI compatible surgical suction instruments have been proposed or utilized that are malleable and are available with various tip configurations.

The single use instruments of the invention are MRI compatible, lightweight, low in cost and have sufficient rigidity to perform the required surgical tasks. In one embodiment, the instruments are constructed of polymeric materials, graphite, titanium, a nitonol composition or combinations thereof. The instruments are constructed for single or short-term usage and are provided to be either sterile or non-sterile instruments. In another embodiment, the instruments are constructed of polymeric materials such as acrylic, nylon and the like to provide a transparent view of the surgical site beneath the instrument. These instruments may also include both sterile or non-sterile instruments. The surgical instruments may also have a non-transparent surface or portions of the instrument may be constructed of combinations of transparent and non-transparent materials, the latter including nitinol compositions and the like.

### SUMMARY OF THE INVENTION

The present invention provides surgical and microsurgical instruments which are MRI compatible and which are constructed and arranged to provide low cost, single use surgical instruments.

The present invention provides surgical instruments which are constructed of MRI compatible materials including but not limited to polymeric substances, polycarbonate, nylon, acrylics, Delrin®, titanium (commercially pure or similar medical grade alloys), nitonol, graphite composite materials or like materials and combinations thereof, and which permit use of these instruments in an interventional MRI suite.

One embodiment of the surgical instruments of the present invention is comprised of an elongated body having a handle portion and a working end portion. Preferably the surgical instruments are formed of a MRI compatible material and may have a portion constructed of a transparent material. The handle portion is preferably constructed of a graphite composition and the working end portion is preferably constructed of titanium or anodized titanium. The handle portion may further comprise a reference point, for example an indentation or a protrusion or the like to thereby provide a location reference for the surgeon. The handle portion may further be textured for example by having patterns or grooves in the handle portion surface. The handle portion of the instruments may also have a low durometer gripping surface, for example, a coating, a polyolefin shrink wrap tubing, a rubber material or the like. The surgical instruments have working end portions having various configurations and having tips disposed at an angle, for example, 0°, 30°, 60°, 80° or other predetermined angles.

The surgical instruments may have clear transparent polymeric portions to provide a clear view of the surgical site beneath and through the instrument structure. The surgical instruments may also be non-reflecting to prevent light, glare or reflection while the instrument is being utilized, for example, by using a different material or by dulling the surface of the instruments.

Another embodiment of the surgical instruments relates to surgical suction instruments which are MRI compatible and designed for single use. The instruments comprise a suction hub member and a length of tubing for suction. The tubing may be straight or pre-bent and may have various combinations including a straight tip, a suction tip, a teardrop tip member, a ported tip member, a medium tip member, or the like. The teardrop tip member and the medium tip member provide depth penetration control while the ported tip member provides greater surface area to draw suction from the surgical site. Preferably the suction hub member is constructed to provide for adjustable suction, for example, by having a teardrop shaped cutout in the front or top face of the suction hub member and has a barbed connection to fit standard size suction tube diameters.

The surgical suction instruments of the present invention preferably have suction tubing which are malleable to allow for bending to a preferred or specified angle to thereby remain out of the field-of-view of the user. The tubing may be provided in a straight or pre-bent configuration. For malleability, annealed or seamless titanium or nitonol tubing may be used in these surgical suction instruments. The surgical suction instruments are also constructed with a variety of tip configurations, i.e., straight or standard, medium, ported and teardrop, to thereby provide for various suction requirements.

The instruments of the invention are constructed to provide low cost, single or multi-use instruments to the physician. The surgical instruments may be provided in a sterile condition, for example, in a sealed tray or envelope for use. The single use or non-reuse feature of these instruments aids in the prevention of the transmission of potentially fatal diseases to the patient. The instruments are lightweight and have sufficient rigidity to perform surgical procedures. A carbon graphite shaft may be used for the instrument handle or body to provide rigidity, lightweight and MRI transparency. Further, the instruments may have various handle portion configurations and may be constructed and arranged to utilize interchangeable tips or working ends, for example, probes, dissectors, ring curettes and the like.

In summary, the present invention provides surgical instruments which are designed for single use, which are MRI compatible and which allow a clear view of the surgical site beneath and through the instrument by utilizing a body construction which is transparent or clear. Other instrument embodiments are provided using non-reflecting or a non-transparent material to prevent reflection or glare from the instrument body while a physician is operating on a patient.

It is an advantage of the instruments of the invention to be constructed of MRI compatible materials to thereby allow the use of the instruments in an interventional MRI suite. The instruments are constructed of a rigid and lightweight material, such as formed plastics, alloys or the like, and combinations thereof.

Another advantage relating to the instruments of the invention is that they are constructed and arranged to provide low cost, single use surgical instruments to the physician. The non-reuse and disposability of these instruments aid in the prevention of the transmission of potentially fatal diseases to a patient, for example.

Further advantages of the surgical instruments of the present invention are their transparency, non-glare features and low cost construction, thereby providing an effective disposable instrument which may be disposed of via incineration, for example.

These and other benefits and advantages of this invention will become clear from the following description by reference to the drawings.

### BRIEF DESCRIPTION OF THE DRAWINGS

FIG. 1 is a top plan view of a surgical probe instrument according to the teachings of the invention;

FIG. 2 is a top plan view of a dissector instrument of the invention;

FIG. 3 is a lateral view of the dissector instrument of FIG. 2;

FIG. 4 is a top plan view of a ring-curette instrument of the invention;

FIG. 5 is a top plan view of another surgical probe instrument;

FIG. 6 is a lateral plan view of a surgical instrument having a bayonet style handle;

FIG. 7 is a top plan view of a textured handle member for an instrument of the invention;

FIG. 8 is a top plan view of another textured handle member for an instrument of the invention;

FIG. 9 is a top plan view of a suction instrument of the invention;

FIG. 10 is a top plan view of another surgical suction instrument;

FIG. 11 is a top plan view of another surgical suction instrument;

FIG. 12 is a top plan view of another surgical suction instrument; and

FIG. 13 is a top plan view of another surgical suction instrument.

### DESCRIPTION OF THE PREFERRED EMBODIMENTS

The present invention provides a variety of surgical and microsurgical instruments which are designed for single use. The instruments are constructed to be used as surgical and microsurgical instruments known and familiar in the surgical industry, however, they may be designed to be of a clear or a non-reflecting material and are designed for one time use. For example, the surgical instruments may be constructed of a rigid, strong plastic material and/or various alloys and combinations thereof as will be further described herein.

The surgical instruments of the present invention are designed for neurosurgery, orthopedic surgery as well as other surgical procedures. For example, the instruments may include probes, micro-ring curettes, micro-probes, dissectors, suction instruments and the like. The surgical instruments are constructed of rigid, lightweight, low cost materials which are transparent and/or of non-reflecting materials and which are also MRI compatible.

The surgical instruments are designed for single use and to be disposable to prevent the transmission of disease from patient to patient, however, it is also within the purview of this invention that the instruments be reused, for example, on a short term basis or for multiple uses. The instruments are preferably provided in packaging to maintain the sterility of the instruments, for example, in a sealed tray or a pouch, i.e., made of Tyvek® or a like material. The instruments further are provided with a ridge, indentation or other marking to provide a reference point for the hand of the user so as to permit the instrument to be held in a known, neutral position during use. The reference point on the instrument permits the surgeon, for example, to position the instrument by feel in the hand without actually requiring viewing of the instrument in the hand.

The instruments of the invention are constructed to provide low cost, single or multi-use instruments to the physician. The single use or non-reuse nature of these instruments aids in the prevention of the transmission of potentially fatal diseases to the patient. The instruments have sufficient rigidity to perform surgery while being lightweight. A carbon graphite shaft may be used for the instrument handle or body to allow rigidity, lightweight and MRI transparency. A titanium or anodized titanium working end may be provided for clarity in an MRI scan and cleanliness in a surgical process.

The surgical instruments include suction instruments which are preferably malleable to allow for bending of the suction tubing to a preferred or specified angle to remain out of the field-of-view of the physician. In order to provide for malleability, these surgical suction instruments use annealed or seamless, titanium or nitonol tubing. The surgical suction instruments are also constructed with a variety of tip designs, i.e., straight or standard, medium and teardrop, to thereby provide for various suction requirements.

The surgical instruments may further provide a clear view of the surgical site beneath and through the instrument structure by utilizing transparent clear materials in their construction. The instruments may also utilize a non-reflecting surface or a non-transparent material to prevent reflection or glare while the instrument is being utilized, i.e., the utilization of a non-polished or a rubbed or dulled titanium composition.

The present invention provides surgical instruments which are constructed of MRI compatible materials including but not limited to polymeric materials, polycarbonate, nylon, acrylics, Delrin®, titanium (commercially pure or similar medical grade alloys), nitonol, graphite composites or like materials and combinations thereof, and which permit use of these instruments in an interventional MRI suite.

Delrin® is an acetal resin polymer manufactured by DuPont™. The polymer is a lightweight, durable, low wear and low friction plastic. Nitinol or nitonol is a nickel-titanium shaped memory alloy. Nitinol alloys may have compositional variations and may be work hardened and heat treated to effect strength and shape memory properties. The alloys are corrosion resistant, non-magnetic, moderately impact and heat resistant, biocompatible and biodegradable. Interventional Magnetic Resonance Imaging (iMRI) involves the application of MRI to guide and monitor generally minimally invasive procedures, such as electrode placement and MRI guided biopsy.

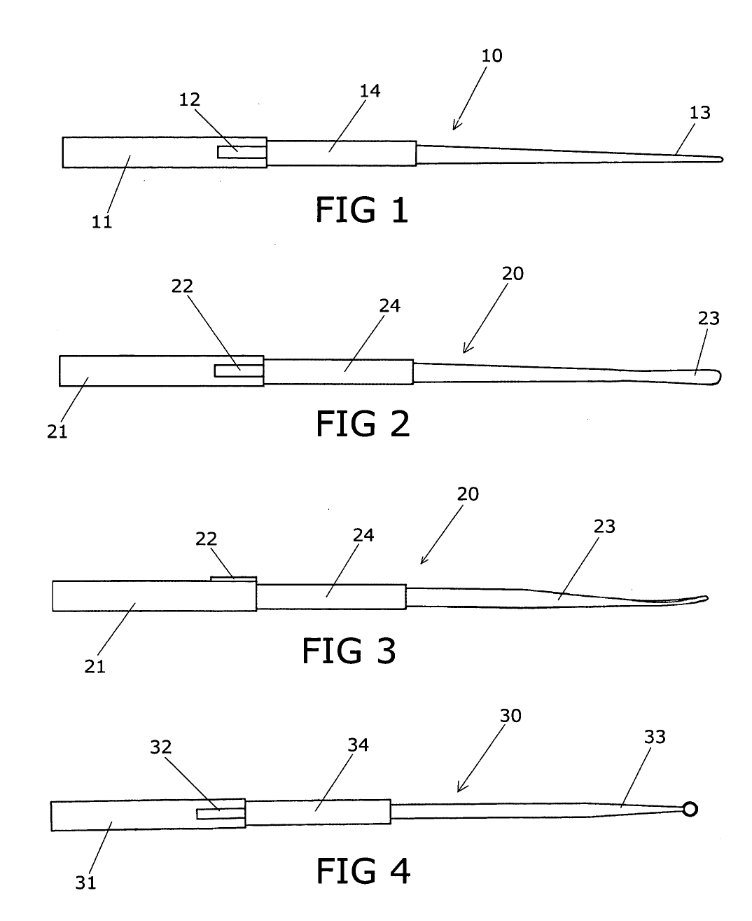

Referring to FIGS. 1-13, various surgical tools are shown according to the teachings of the present invention. FIG. 1 shows surgical probe instrument 10 which has handle portions 11 and 14 and working end 13. Reference point 12 is shown disposed on handle portion 11 of probe instrument 10. Reference point 12 may be an indentation, a protrusion or the like in handle portion 11 of the instrument. The surgical probe of FIG. 1 is exemplary. Importantly, the probe or portions of the probe, i.e., handle portion 14 may be constructed of a transparent material such as a plastic, for example. Further, the instrument may be formed of a low cost, rigid material having a non-glare surface, or the non-glare surface may be formed by dulling the material, i.e., titanium. The latter instrument may also be constructed of a non-transparent structure or have portions which are non-transparent. For example, as shown, the handle portion of the probe instrument may be constructed of a polymeric material or graphite and which is connected to a tip or working end that may be constructed of a different material composition, for example titanium. The tip or working end may also be interchangeable with the handle portion, i.e., frictionally connected or fastened in place using an adhesive such as an epoxy or the like, as will be further described with respect to FIGS. 7 and 8. Handle front portion 14 may be constructed of graphite along with the handle portion 11 or may be formed of a transparent polymeric material.

The surgical instruments of FIGS. 1-5 are each shown having an elongated straight handle member having two handle portions. For example, referring to FIG. 1, the handle member is shown to have a first handle portion 11 and a second handle portion 14 to which the working portion 13 of the instrument is connected. The handle member is generally a cylindrical structure, i.e., ¼ inch diameter carbon graphite rod which is machined to form the handle member of the instrument. The first handle portion 11 has a first diameter and the second handle portion 14 has a second diameter which is smaller than the first diameter. The reference point 12 is shown axially aligned on or in the first handle portion at the diameter step down to the second handle portion. The first and second handle portions may be a one piece generally cylindrical graphite structure or may be a structure of two or more different compositions, i.e., a first handle portion constructed of graphite and the second handle portion constructed of a clear polymeric material.

FIGS. 2 and 3 show front and side views, respectively, of an exemplary dissector instrument 20, portions of which may also be constructed of a transparent material composition or of a formed material having a non-glare surface. The instrument is also shown to have dissector working or tip end 23 and reference point 22 on handle portion 21 for use by the surgeon, for example. Reference point 22 is shown protruding from handle portion 21 and aligned axially on the instrument 20. Handle front portion 24 is shown extending from handle portion 21. Working tip end 23 is shown extending from handle front portion 24 and may be constructed of various compositions, as desired. The handle portions are preferably constructed of graphite, however, handle front portion 24 may alternatively be constructed of a transparent material to provide a view of the surgical site.

FIG. 4 shows exemplary ring curette instrument 30 having handle portion 31 with reference point 32, handle front portion 34 extending therefrom and a ring curette working end 33 extending from portion 34. The reference point 32 may be an indentation or protrusion or the like.

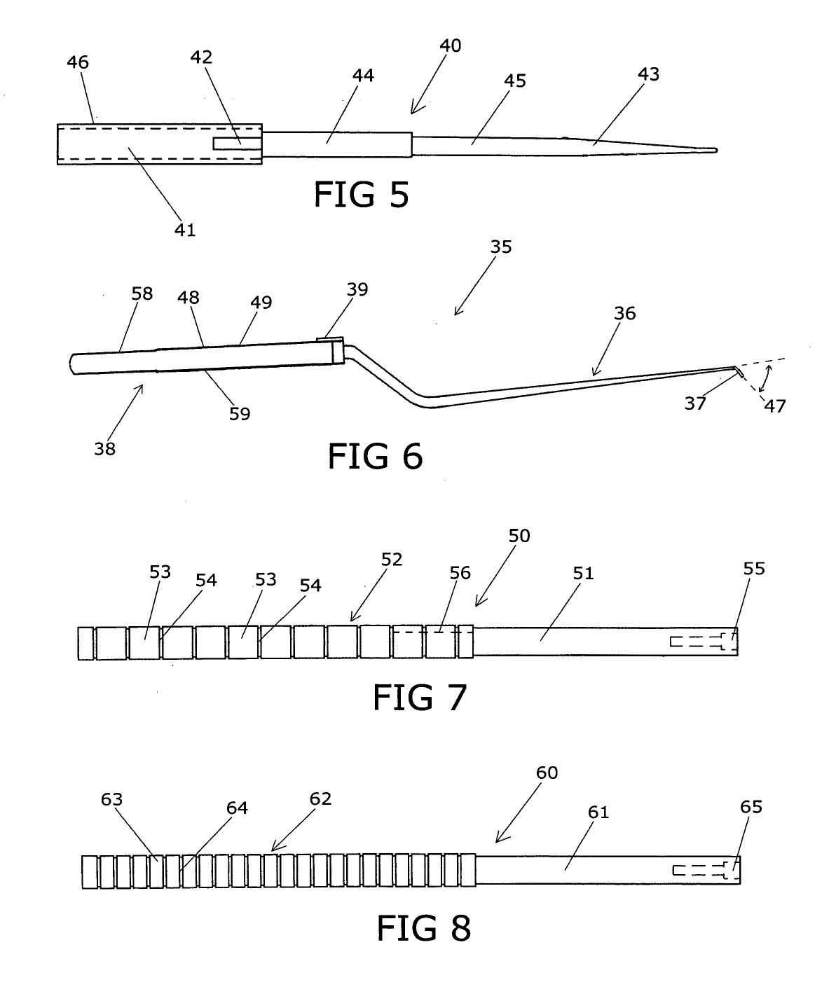

FIG. 5 shows a probe instrument 40 having handle portion 41 with reference point 42 which may be constructed of a polymeric material having graphite reinforcement, for example. Handle front portion 44 is shown extending from handle portion 41 and connected to working end 43 and which may be transparent, thus allowing visibility at the work site. Working end 43 is shown having anodized surface 45 for better MRI clarity.

FIG. 6 shows surgical instrument 35 having working end 36, bayonet style handle portion 38 and reference point 39. Handle portion 38 is shown comprised of first handle portion 48 and second handle portion 58, where first handle portion 48 is adjacent and stepped up from second handle portion 48 and is adjacent to working end portion 36. First handle portion 48 is shown to have opposing flat surfaces 49 and 59 for ease of handling by a surgeon. Surgical instrument 35 is shown having a bent portion for ease of use of the surgeon. Working end 36 is shown having tip member 37 disposed at an angle of approximately 60°. Tip member 37 may be disposed at angle range 47, i.e., approximately 0-90°.

The probe, dissector and ring curette instruments of FIGS. 1-6 may have their respective tip or work portions and/or handle portions secured via an adhesive or other securing means to thereby permit the handle portion and the working tip portion to be constructed of different materials, i.e., graphite reinforced handle portions and a polymeric or titanium working tip portion.

As shown in FIG. 5, the handle portions of the surgical instruments may have a low durometer covering or coating 46 to create a soft handle portion surface for ease of use and ergonomics. The low durometer covering material may be polyolefin heat shrunk tuning, soft rubber or a like material. The tips of the working end portions of FIGS. 1-6 may further be disposed at an angle to aid in the surgical process. For example, the tips of the working end portions may be disposed at 0°, 30°, 60°, 80° or at any predetermined angle.

FIGS. 7 and 8 show handle members 50 and 60 having handle bodies 51 and 61 with textured handle portions 52 and 62 respectively, which may be used with the surgical instruments of FIGS. 1-6. The working ends of the surgical instrument shown in FIGS. 1-6 may be attached to handle member 50 or 60 using connecting ends 55 and 65 whereby the working ends have matching connecting ends that are press fit into the connecting apertures 55 and 56 of the handle members 51 and 61. The textured handle portions 52 and 62 are shown to have patterns of grooves 54 and 64, respectively, to thereby form protrusions 53 and 63 to aid in the ergonomic handling and use of the instrument. The textured handle portions may be formed into the graphite or a like material which make up the handle member.

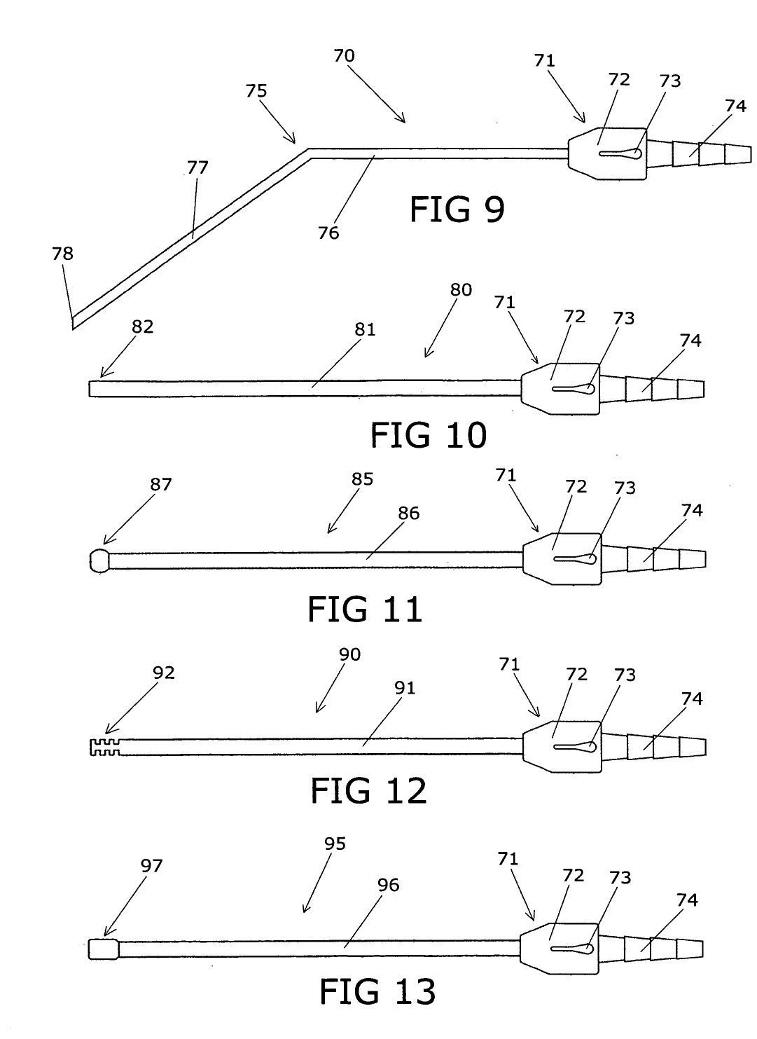

FIG. 9 shows a surgical suction instrument 70 which may also be constructed of a clear, polymeric material and designed for one time use. Surgical suction instrument 70 is shown comprising section hub member 71 and tubing 75 comprising tubing portions 76 and 77 and tubing tip 78. The suction instrument is merely exemplary and may be constructed in various sizes and configurations. The suction hub member 71 of instrument 70 is shown to have a keyhole or teardrop style opening 73 for suction control and a barbed connection 74 for attachment. The hub member 71 may be constructed of a polymeric or titanium material, whereas the tubing 75 may be constructed of a nitinol composition, for example. FIGS. 10-13 show various tip members 82, 87, 92 and 97 of tubing 81, 86, 91 and 96 that may be used with surgical suction instruments 80, 85, 90 and 95.

FIG. 10 shows a surgical suction instrument 80 having a standard or straight tip 82 of tubing 81. FIGS. 11, 12 and 13 show surgical suction instruments 85, 90 and 95, respectively, having teardrop tip member 87, ported tip member 92 and medium tip member 97, respectively. Teardrop tip member 87 and medium tip member 97 aid in preventing depth penetration while performing suction at the surgical site, while ported tip member 92 provides greater surface area for suction purposes.

Surgical suction instrument hub member 71 is preferably constructed and arranged having hub body 72, a teardrop shaped cutout 73 in a face of the hub body and a barbed connection 74 to fit standard size suction tubing sizes. Further, various tubing structures may similarly be utilized, i.e., nitinol tubing having different lengths, diameters and configurations.

Alternatively, the handle portions of the instruments of the invention may be constructed to allow for interchangeable tips or working ends. For example, the respective embodiments of the straight handle portion of FIGS. 1-5, the bayonet handle portion of FIG. 6, and the textured handle portions of FIGS. 7 and 8 may be utilized interchangeably with the various working ends shown in FIGS. 1-6, i.e., the dissector, probe and ring curette working ends. For example, the tips may have different compositions, different sizes and configurations and may be utilized with a single disposable handle to which the tip or ends may be secured and removed, i.e., via a frictional fit, for example.

As many changes are possible to the embodiments of the instruments of this invention, utilizing the teachings thereof, the description above and the accompanying drawings should be interpreted in the illustrative and not the limited sense.

That which is claimed is:

1. In a surgical instrument having an elongated body having a handle member and a surgical working portion extending therefrom, the improvement comprising said elongated body being formed of an MRI compatible composition and said handle member having a reference member positioned with respect to the handle member for the hand of the user.

2. The surgical instrument of claim 1, wherein said MRI compatible composition is selected from the group of compositions consisting of polymeric materials, graphite, titanium, nitinol and combinations thereof.

3. The surgical instrument of claim 2, wherein said handle member is comprised of a generally cylindrical structure having a first handle portion with a first diameter and a second handle portion with a second diameter, said first diameter being larger than said second diameter, wherein said reference member positioned with respect to said first handle portion and wherein said working portion extends from said second handle portion.

4. The surgical instrument of claim 3, wherein said first handle portion is formed of graphite, wherein said second handle portion is formed of a polymeric material and wherein said working portion is formed of titanium.

5. The surgical instrument of claim 4, wherein said reference member is an elongated protrusion member extending axially from said first handle portion or an elongated indentation member extending axially in said first handle portion.

6. The surgical instrument of claim 2, wherein said handle member has a textured surface.

7. The surgical instrument of claim 6, wherein said handle member has a polyolefin heat shrinked tubing or a rubber material thereabout, each having a low durometer reading.

8. The surgical instrument of claim 2, wherein said handle member is a bayonet style handle member.

9. The surgical instrument of claim 1, wherein said working portion is formed of a material selected from the group of materials consisting of nitinol, polycarbonate, nylon, acrylic, acetal resin polymer, titanium, graphite and combinations thereof.

10. The surgical instrument of claim 1, wherein said working portion is comprised of titanium and wherein said titanium is anodized.

11. The surgical instrument of claim 1, wherein said working portion has a terminal connecting member, wherein said handle member has a terminal connecting member and wherein said connecting members of said handle member and said working portion are press fit to form said surgical instrument.

12. A surgical instrument for use in interventional MRI suites comprising a body member formed of a material having MRI transparency, said material selected from the group of materials consisting of polymeric materials, graphite, titanium, a nitonol composition and combinations thereof.

13. The surgical instrument of claim 12, wherein said body member includes a handle member having a reference point.

14. The surgical instrument of claim 13, wherein said handle member is generally cylindrical in cross-section and wherein said reference point is an elongated protrusion member or an elongated indentation member extending axially along said handle member.

15. The surgical instrument of claim 14, wherein said handle member has a first portion with a first diameter and a second portion with a second diameter, said first diameter being greater than said second diameter, said first portion having a textured surface.

16. The surgical instrument of claim 14, wherein a polyolefin heat shrinked tubing or soft rubber material is positioned about at least a portion of said handle member.

17. The surgical instrument of claim 13, wherein said handle member is a bayonet style handle member.

18. The surgical instrument of claim 13, wherein said body member includes a working portion and wherein said working portion extends from said handle member.

19. The surgical instrument of claim 18, wherein said handle member is constructed of graphite and wherein said working portion is formed of a material selected from the group of materials consisting of nitinol, polycarbonate, nylon, acrylic, acetal resin polymer, titanium and combinations thereof.

20. The surgical instrument of claim 19, wherein said working portion is comprised of titanium and wherein said titanium is anodized.

21. The surgical instrument of claim 19, wherein said instrument comprises a probe, dissector or ring-curette and has an angled distal working portion tip in a range of 0° to 90°.

22. The surgical instrument of claim 12, wherein said instrument is a surgical suction instrument having a hub and a malleable suction tube, said suction tube being formed of nitinol or titanium.

23. A surgical instrument for use in conjunction with a magnetic resonance imaging procedure, said surgical instrument comprising:
a) an elongated handle portion, said elongated handle portion being constructed and arranged having a reference point; and
b) a working end portion extending from said elongated handle portion, wherein said working end portion is made of a magnetic resonance imaging transparent material and which is compatible with magnetic resonance imaging procedures.

24. The surgical instrument of claim 23, wherein said reference point is an indentation or a protrusion on or in said elongated handle portion.

25. The surgical instrument of claim 23, said working end portion has an angled distal end.

26. The surgical instrument of claim 25, wherein said angled distal end ranges between approximately 0-90°.

27. The surgical instrument of claim 23, wherein said handle portion is formed of a generally cylindrical body constructed of graphite.

28. The surgical instrument of claim 23, wherein said handle portion is textured and wherein said texture is provided by parallel grooves in said handle portion.

29. The surgical instrument of claim 23, wherein said handle portion includes a covering.

30. The surgical instrument of claim 29, wherein said covering is made of a material selected from the group of materials consisting of polyolefin and rubber.

31. The surgical instrument of claim 23, wherein said magnetic resonance imaging transparent material of said working end portion is titanium or anodized titanium.
